(19) United States
(12) Patent Application Publication
(10) Pub. No.: US 2021/0177897 A1
(43) Pub. Date: Jun. 17, 2021

(54) ARGINASE SUPPRESSION FOR CANCER TREATMENT

(71) Applicant: Université de Genève, Geneve 4 (CH)

(72) Inventors: Thibaut De Smedt, Prevessin-Moens (FR); Walter Reith, Vessy (CH); Adria-Arnau Marti Lindez, Geneve (CH); Isabelle Dunand-Sauthier, Geneve 4 (CH)

(21) Appl. No.: 16/963,380

(22) PCT Filed: Jan. 24, 2019
(86) PCT No.: PCT/EP2019/051806

§ 371 Date: Jul. 20, 2020

(30) Foreign Application Priority Data
Jan. 28, 2018 (EP) 18153782.0

(51) Int. Cl. A61K 35/17; A61P 35/00; A61K 38/17; A61K 39/395; C12N 15/113

(52) U.S. Cl. CPC A61K 35/17; A61P 35/00; A61K 2039/505; A61K 39/3955; C12N 15/1137; A61K 38/1774

(57) ABSTRACT

The present invention concerns a method for treating cancer, including haematological and solid tumors. In an embodiment, the method comprises impairing arginase activity and/or expression in immune cells, in particular T cells of a patient suffering from cancer. Arginase expression may be impaired by mutation (including deletion or truncation) of the arginase encoding gene, by RNA interference or by administration of an arginase inhibitor. In a preferred embodiment, the T cells are modified in the frame of CAR (Chimeric Antigen Receptor) therapy. The invention also provides a method of treatment combining impaired arginase activity with antibody-mediated blockage of negative immune checkpoint regulators (PDLL-PD1 and B7-CTLA4 inhibitory pathways).

### TECHNICAL FIELD

The present invention relates to immune cells, kits, methods and compositions for use in the treatment of cancer, in particular in the field of cancer immunotherapy and/or adoptive cell transfer therapy. The invention also relates to methods for producing the cells, kits and compositions.

### BACKGROUND ART AND PROBLEMS SOLVED BY THE INVENTION

The immune system plays fundamental roles in suppressing the initiation of malignant neoplasms, inhibiting tumor progression and promoting tumor elimination. With the aim of escaping immune surveillance, tumors tend to implement diverse evasion mechanisms. Immune evasion strategies include the production of anti-inflammatory cytokines, the recruitment of inhibitory cells comprising regulatory T cells (T_{regs}) and myeloid-derived suppressor cells (MDSCs), the expression of negative T-cell co-stimulatory molecules, and the activation of immunosuppressive metabolic pathways. Boosting tumor-directed immune responses by inhibiting such evasion mechanisms is a strategy that bears significant therapeutic promise. Antibody-mediated blockade of negative immune checkpoint regulators is notably one of the most promising and successful immunotherapeutic approaches so far. Such immune checkpoint pathways normally prevent or mitigate immunopathology by terminating or dampening excessive T-cell responses. Tumor cells exploit this mechanism to prevent attack by T cells. Releasing these “brakes” by means of therapeutic intervention can thus restore effective anti-tumor immune responses. However, despite unprecedented successes obtained by interfering with the PDLL-PD1 and B7-CTLA4 inhibitory pathways, current checkpoint blockade therapies do not elicit effective anti-tumor immune responses in all patients, are not active against all types of cancer, have only partial efficacy, or tumors develop resistance to checkpoint blockade.

Another form of cancer immunotherapy consists of utilizing the patients' immune cells to eradicate cancer via adoptive cellular therapy, either in the form of ex vivo expansion of a patient's tumor-infiltrating lymphocytes (TILs) and reinfusion of a population of activated T lymphocytes responsive to tumor antigens, or removal of a patient's peripheral blood T lymphocytes and ex vivo genetic modification to express a transgenic T cell receptor (TCR) or a chimeric antigen receptor (CAR) to target a known tumor antigen, followed by reinfusion into the patient to destroy cancer cells. CAR T cell immunotherapy has shown unprecedented success in haematological tumours, but treatment of solid tumours using CAR T cells has been largely unsuccessful so far, in part due to the immunosuppressive tumor micro-environment. In addition, considerable attention is required to prevent excessive exhaustion or differentiation of the CART cells during in vitro manufacturing.

There is thus an unmet need for the development of new strategies aimed at inducing potent anti-tumor responses, alone or in combination with existing therapies. In particular, synergistic treatment combinations involving different immunotherapeutic agents, together with other complementary therapies, might constitute the basis of successful anti-cancer treatments in the future.

One therapeutic strategy that merits further investigation is interfering with metabolic pathways that favor tumor growth by inhibiting anti-tumor immune responses. Among these, increasing evidence suggests that therapeutic manipulation of L-Arginine metabolism could help to boost anti-tumor immune responses. L-Arginine is a semi-essential amino acid that needs to be assimilated from the extracellular environment by certain cells, notably certain cells of the immune system, including T cells and macrophages. Sufficient L-Arginine availability is essential for optimal function of these cells. In the case of T cells, L-Arginine depletion results in reduced expression of the CD3-ζ chain, impaired signaling via the T cell receptor (TCR)-CD3 complex, and suppression of antigen-specific T-cell activation, proliferation and cytotoxicity.

L-Arginine is a substrate for arginases (ARGs) and nitric oxide synthases (NOS). L-Arginine catabolism by NOS leads to the production of NO and other reactive nitrogen intermediates, which is a key mechanism contributing to the cytotoxic activity of macrophages against pathogens. ARGs convert L-Arginine to urea and L-Ornithine, a reaction best known for its role in detoxification of ammonia in the liver by the urea cycle. L-Ornithine is further processed to polyamines and proline. The expression of ARG and NOS enzymes has been found to be increased in tumors, either in the tumor cells themselves or in tumor-infiltrating cells such as macrophages and myeloid-derived suppressor cells (MDSCs). In the tumor micro-environment (TME), NOS-mediated NO production promotes tumor angiogenesis and metastasis, whereas ARG-mediated increases in proline availability and polyamine synthesis promote tumor-cell proliferation. Furthermore, L-Arginine depletion induced by ARG and NOS expression in tumors contributes to creating an immunosuppressive micro-environment that impairs anti-tumor T cell responses by the mechanism outlined above.

Mammals have two ARG enzymes, arginase-1 (ARG1) and arginase-2 (ARG2), encoded by two different genes. The two enzymes catalyze the same biochemical reaction, exhibit 100% homology in their catalytic sites, and are both inhibited by the currently available ARG inhibitors. ARG1 and ARG2 differ with respect to their intracellular localization and pattern of expression. ARG1 is a cytosolic enzyme expressed predominantly in the liver, but also in several non-hepatic tissues and cell types. ARG2 is located in mitochondria, and exhibits widespread expression in diverse tissues.

In the publication by Dunand-Sauthier I. et al, “Repression of arginase-2 expression in dendritic cells by microRNA-155 is critical for promoting T cell proliferation”, J Immunol. 193:1690-1700 (2014), ARG2 was found to be the dominant arginase expressed in dendritic cells (DCs) and its corresponding mRNA was shown to be repressed by miR155 during DC maturation. Abnormally elevated levels of ARG2 expression and activity were observed in activated miR155-deficient DCs. Conversely, overexpression of miR155 inhibited ARG2 expression in DCs. miR155 is well known to control different genes in different cell types, and it has not been reported whether miR155 also controls ARG2 expression in other cell types, including T cells.

WO2014059248 teaches a method of increasing T cell mediated immunity by introducing ex vivo a nucleic acid molecule encoding a miR155 transcript into T cells isolated from a subject. US2015/275209 similarly teaches isolated or purified CD8+ T cells comprising an antigen-specific T cell receptor (TCR) and an exogenous nucleic acid encoding a microRNA-molecule. Neither WO2014059248 nor US2015/275209 disclose the manner of the activity of miR-155 in T cells. In the publication by Gracias D. et al, “MicroRNA-155 controls CD8⁺ T cell responses by regulating interferon signaling”, Nat Immunol. 14:593-602 (2013), miR155 was found to modulate the expression of 845 genes in CD8⁺ T cells, the majority of which showed less than 2-fold differential expression, suggesting that miR155 moderately affects large numbers of transcripts, rather than having a robust effect on individual targets. Gracias et al. does not identify ARG2 among the 845 genes the expression of which is modulated by miR155. These references do thus not suggest that ARG2 could be a target of miR155 in T cells and that the effect of miR155 is mediated via ARG2 inhibition in T cells.

Several patent documents are concerned with arginase inhibitors. EP2768491B1 discloses small molecule arginase inhibitors for treating a number of conditions, such as cardiovascular disorders, sexual disorders, wound healing disorders, gastrointestinal disorders, autoimmune disorders, immune disorders, infections, pulmonary disorders, fibrotic disorders and haemolytic disorders. EP2083812B1 discloses small molecule arginase inhibitors, such as 6-borono-L-norleucine (ABH) for treating allergic and non-allergic asthma as well as allergic rhinitis. WO2007/111626 discloses the modulation of ARG2 activity for the treatment or atherosclerotic disease, including siRNA for silencing ARG2. U.S. Pat. No. 9,789,169 discloses recombinant ARG1 proteins for depleting the plasma arginine levels, with the goal of modulating the immune system, in particular preventing rejection of a transplanted organ.

It is an objective of the present invention to provide a treatment for cancer, which is preferably effective against hematopoietic neoplasms and/or solid tumors.

It is an objective of the invention to improve the efficacy of therapies based on chimeric antigen receptor (CAR) T cells and/or a transgenic T cell receptor T cells, and/or of therapies based on interactions with and/or blocking of immune checkpoint regulator(s). For example, it is an objective to provide a way of improving existing cancer therapies or existing therapeutic concepts.

It is an objective of the present invention to provide a treatment for cancer that may be synergistically used with other cancer treatments, including immunotherapeutic agents.

It is an objective of the present invention to treat cancer by promoting cancer-directed immune responses. In particular, it is an objective of the present invention to inhibit mechanisms that cancer cells exploit for evading cancer-directed immune responses.

The present invention addresses the needs and objectives described above.

### SUMMARY OF THE INVENTION

Remarkably, the present inventors conducted experiments showing that immune cells having impaired arginase activity and/or expression are more efficient for the treatment of cancer, including solid tumors.

In an aspect, the present invention provides immune cells having impaired arginase activity and/or expression for treating cancer, including solid tumors.

In an aspect, the present invention provides immune cells expressing a chimeric antigen receptor (CAR), said immune cells further having impaired arginase activity and/or expression.

In an aspect, the present invention provides tumor infiltrating lymphocytes (TILs) having impaired arginase activity and/or expression. Preferably, said TILs have been isolated from a human or animal suffering from cancer.

In an aspect, the present invention provides immune cells exhibiting constitutively or inducibly impaired arginase activity and/or expression for treating cancer by adoptive cell transfer.

In an aspect, the present invention provides a method for treating cancer, the method comprising impairing arginase activity and/or expression in immune cells.

In an aspect, the present invention provides a method for treating cancer comprising administration, to a subject in need thereof, of immune cells having impaired arginase activity and/or expression. Preferably, the method is a method for treating cancer by adoptive cell transfer.

In an aspect, the present invention provides a method for treating cancer, the method comprising administrating tumor infiltrating lymphocytes (TILs), said TILs further having impaired arginase activity and/or expression.

In an aspect, the present invention provides a method for treating cancer, the method comprising administrating immune cells expressing a CAR, said immune cells having impaired arginase activity and/or expression.

In an aspect, the present invention provides a method for treating cancer, the method comprising: providing cells expressing a CAR and/or TILs, treating said cells, preferably in vitro and/or ex vivo so as to reduce arginase activity and/or expression in said cells, and administrating said cells to a subject in need thereof, preferably a subject suffering from cancer.

In an aspect, the present invention provides a method for improving the anti-cancer activity of an immune cell, the method comprising: impairing arginase activity and/or expression in said immune cell, preferably impairing ex vivo the arginase activity and/or expression in said immune cell.

In an aspect, the present invention provides a method for improving the anti-cancer activity of immune cells for adoptive cell transfer, the method comprising: impairing arginase activity and/or expression in said immune cell, preferably ex vivo.

In an aspect, the present invention provides a method for preparing and/or manufacturing an anti-cancer treatment, in particular for adoptive cell transfer, the method comprising:

providing immune cells, and impairing ex vivo the arginase activity and/or expression in said immune cells.

In an aspect, the present invention provides an agent capable of reducing arginase activity in immune cells for treating cancer.

In an aspect, the present invention provides a kit for treating cancer, the kit comprising an agent suitable for impairing arginase activity and/or expression in an immune cell.

In an aspect, the present invention provides a combination treatment comprising administering immune cells having impaired arginase activity and/or expression and an agent blocking an immunosuppressive pathway, such as the PDLL-PD1 and B7-CTLA4 inhibitory pathways. Preferably, said agent is an antibody (Ab), for example an anti-PD1 Ab or an anti-CTLA4 Ab.

In an aspect, the present invention provides a composition comprising immune cells, in particular the immune cells of the invention.

In an aspect, the present invention provides immune cells having impaired arginase activity and/or expression for reducing tumor size and/or volume in subjects suffering from a tumor and/or cancer. In an aspect, the present invention provides immune cells having impaired arginase activity and/or expression for increasing survival rate and/or time in subjects suffering from cancer.

In an aspect, the present invention provides a method for producing a treatment for cancer, the method comprising providing an isolated immune cell and treating said immune cell so as to have impaired arginase activity and/or expression.

Further aspects and preferred embodiments of the invention are defined herein below and in the appended claims. Further features and advantages of the invention will become apparent to the skilled person from the description of the preferred embodiments given below.

### BRIEF DESCRIPTION OF THE FIGURES

In the figures, results of the experimental section are shown for the purpose of illustrating embodiments and/or examples of the present invention.

FIGS. 1A and 1B show tumor growth in WT and Arg2^{-/-} mice with implanted B16-OVA or MC38-OVA tumor cells. The figures show that tumor growth was impaired in the Arg2^{-/-} mice.

FIGS. 2A and 2B show anti-tumor immunity in mice as described for FIGS. 1A and 1B. The figures show that anti-tumor immunity was enhanced in the Arg2^{-/-} mice.

FIG. 3A shows MC38-OVA tumor growth in WT (circles) and Arg2^{-/-} (squares) mice, which were (clear circles and squares) or not (filled circles and squares) depleted of CD8⁺ T cells. In these experiments, CD8⁺ cell depletion increased tumor growth.

FIG. 3B shows decreased animal survival of the mice depleted of CD8⁺ cells described in FIG. 3A.

FIGS. 4A, 4B and 4C show MC38-OVA tumor growth, tumor clearance and animal survival, respectively, in WT (FIG. 4A: circles) and Arg2^{-/-} (FIG. 4A: squares) mice receiving (FIG. 4A: clear circles and squares) or not (FIG. 4A: filled circles and squares) anti-PD1 or isotype control Abs on days 9, 11 and 14 after tumor injection (arrows). The results show a strong combinatorial effect of anti-PD1 Ab treatment and Arg2-deficiency on tumor size (FIG. 4A: clear squares), tumor clearance and survival.

FIG. 5 shows MC38-OVA tumor growth in chimeric mice generated by reconstituting sub-lethally irradiated WT or Arg2^{-/-} mice with bone marrow (BM) cells derived from WT or Arg2^{-/-} mice in all four pairwise combinations. Results indicate that reduced tumor growth is due primarily to Arg2-deficiency in BM-derived cells. Consequently, mice receiving BM cells from Arg2^{-/-} mice (clear circles and squares) have lower tumor size.

FIGS. 6A and 6B compares in vitro activation (FIG. 6 A) and proliferation (FIG. 6 B) of T cells isolated from OTI WT mice with those from double homozygote Arg2^{-/-} OTI mice, respectively. The results show that Arg2^{-/-} OTI T cells exhibit increased activation and proliferation compared to Arg2^{+/+} OTI T cells.

FIG. 7A illustrates the generation of an in vivo system used for cell therapy in accordance with an embodiment of the invention.

FIG. 7B shows tumor growth in mice that were treated as shown in FIG. 7 A. WT mice receiving Arg2^{-/-} OTI T cells exhibit significantly slower tumor growth.

FIG. 7C shows animal survival of mice that were treated as shown in FIG. 7 A. WT mice receiving Arg2^{-/-} OTI T cells exhibit significantly increased survival.

FIGS. 8A, 8B and 8C show that Arg2-deficient adoptively-transferred CD8+ T cells produce more IFNγ (FIG. 8A), become less exhausted (FIG. 8B) and persist for longer (FIG. 8C) in MC38-OVA bearing WT recipients.

FIGS. 9A, 9B, and 9C show MC-38-OVA tumor volume, animal survival and tumor clearance at day 60, respectively, in WT mice receiving WT OTI T cells (FIG. 9A: circles) or Arg2-deficient OTI T cells (FIG. 9A: squares) and receiving anti-PD1 (FIG. 9A: filled circles and squares) or isotype control Abs (FIG. 9A: clear circles and squares) on days 8, 11 and 14 after T cell transfer (arrows) via i.p. injections. The results show a strong combinatorial effect of anti-PD1 Ab treatment and Arg2-deficiency in adoptive CD8⁺ T cells on tumor volume, survival and tumor clearance.

FIGS. 10A and 10B show that ARG inhibition increases human T cell activation in vitro. Human T cells purified from PBMCs were left untreated (−) or in vitro activated with anti-CD3 and anti-CD28 Abs (+), cultured in RPMI in 96-well plates in the presence or absence of ARG inhibitors, and assessed for activation (CD69 staining) 24 h post-activation. The figures show increased frequency of CD69⁺ cells within CD4⁺ (FIG. 10A) and CD8⁺ T cells (FIG. 10B) in the presence of the indicated ARG inhibitors.

Hereinafter, preferred embodiments of the invention are described, in order to illustrate the invention, without any intention to limit the scope of the present invention.

### DETAILED DESCRIPTION OF THE PREFERRED EMBODIMENTS

In some aspects, the present invention relates to immune cells having impaired arginase activity and/or expression for treating cancer. The cells are preferably used for cancer immunotherapy.

The term “impaired” in the expression “impaired arginase activity and/or expression” is intended to mean an arginase activity and/or expression that is reduced compared to corresponding wild type immune cells that have not been treated, modified and/or engineered, for example as disclosed in the present specification, so as to have said impaired arginase activity and/or expression. In a preferred embodiment, said arginase activity and/or expression is impaired to the extent that no activity and/or expression of the arginase is detectable. Preferably, said arginase activity is completely absent.

The expression “impaired arginase activity and/or expression” encompasses the situation where arginase activity is reduced due to impairment of the transcription and/or translation of the mRNAs encoding arginases. Furthermore, although impaired arginase “activity” and/or “expression” are frequently mentioned together in the present specification, it is understood that impaired arginase expression results in impaired arginase activity and that, therefore, the expression “impaired arginase activity” encompasses the situation where such activity is impaired due to reduced arginase expression. In a preferred embodiment, the immune cell's capacity of producing active arginase is impaired, preferably by technological interference with the immune cell's processes of gene expression—including processes such as transcription, RNA splicing, translation, post-translational modification—in particular the expression of one or more genes encoding arginase.

Said arginase may be arginase 1 (ARG1) and/or arginase 2 (ARG2). In a preferred embodiment, said impaired arginase activity and/or expression is impaired arginase 2 (ARG2) activity and/or expression. As mentioned above, ARG1 and ARG2 differ with respect to their intracellular localization and pattern of expression. ARG2 is located in mitochondria, and exhibits widespread expression in diverse tissues. In an embodiment, activity of both ARG1 and ARG2 is impaired.

In a preferred embodiment, said immune cell is selected from T cells, TILs, natural killer cells (NK cells), innate lymphoid cells (ILC) and dendritic cells. For example, said ILC may be selected from ILC-1 and ILC-2 cells. In a preferred embodiment, said immune cells are T cells that are preferably selected from CD3⁺ and/or CD4+ and/or CD8+ T cells. Combinations comprising two or more different types of immune cells having impaired arginase activity and/or expression may also be used.

In an embodiment, said immune cells are dendritic cells. In an alternative embodiment, said immune cells are not dendritic cells and/or exclude dendritic cells.

For the purpose of the present specification, the term “comprising”, and its various grammatical forms, is intended to mean “includes amongst other”. It is not intended to mean “consists only of”.

In a preferred embodiment, the immune cells are for administration to a subject in need thereof, in particular a subject suffering from cancer. In an embodiment, the present invention relates to cell therapy for treating cancer. Preferably, said immune cells are administered by adoptive cell transfer (ACT) or in the frame of a treatment by ACT. ACT is a treatment of cancer immunotherapy, such as autologous cancer immunotherapy. The term “adoptive” is generally understood to express that ACT is the transfer of cells into a patient. The cells may have originated from the patient or from another individual, for example. In a preferred embodiment, ACT refers to the transfer of cells that have been selected, engineered and/or modified, generally genetically and/or in terms of expressed proteins, so as to render the cells more efficient or apt to treat the condition in question, in general cancer.

In a preferred embodiment, said immune cells are isolated and/or purified.

Said immune cells may originate from an individual that is the patient to be treated. In this case one refers to autologous immunotherapy. In other words, the functionality and characteristics of immune cells of a patient are improved in order to improve their capacity to combat the cancer from which the patient is suffering.

In an embodiment, said immune cells are obtained from the tumor of a patient. In this manner TILs may be obtained.

In another embodiment, the immune cells are taken from a donor. In this case, one may refer to allogeneic immunotherapy.

In yet another embodiment, said immune cells are obtained from stem cells and/or precursor immune cells.

When the immune cells are derived from a patient and/or donor, the method of the invention may comprise collecting and/or extracting said immune cells from said patient or from said donor, in particular from the blood of the patient or donor, or from the tumor of the donor in the case of TILs, for example.

In another embodiment, said cells have been previously collected and/or extracted from an individual, for example from a subject to be treated or from a donor.

If the immune cells are derived from a donor, they are preferably selected or modified so as to not attack healthy tissues in the patient. For example, the immune cells of the donor are preferably compatible with the patient. For example, the donor may be a family member of the patient.

In some embodiments, including whether or not the donor is a family member, the cells of the donor have an inactivated native T cell receptor (in case the immune cells are T cells), so as to prevent the immune cells from attacking healthy patient tissue. The cells are preferably modified and/or engineered not to express the native T cell receptor or to express an inactive form thereof, for example.

As indicated, in some embodiments, the immune cells may be derived from stem cells, for example pluripotent stem cells. Accordingly, the immune cells are preferably prepared from off-the-shelf cells, further engineered in accordance with the present specification. For example, the immune cells may be obtained based on the artificial thymic organoid (ATO) system. The ATO system is an in vitro model that artificially mimics the thymic environment to recapitulate human T-cell development. The ATO system supports efficient differentiation and positive selection of normal T-cells using hematopoietic stem cells from various sources, as well as pluripotent stem cells, like embryonic stem cells and induced pluripotent stem cells. The technology also offers flexibility for further gene engineering to produce off-the-shelf allogeneic engineered T-cell products for therapeutic use, for example. Such technology is disclosed, for example, in WO2016/187459 and WO 2017/075389.

In accordance with the present invention, the immune cells have impaired arginase activity and/or expression. The arginase activity and/or expression in said immune cells may be impaired in any suitable manner. In an embodiment, said arginase activity is impaired by exposure of said immune cells to treatment and/or engineering for impairing said arginase activity and/or expression. Preferably, said treatment is an ex vivo treatment. For example, the cells are exposed to a treatment once extracted from an individual, such as a patient or donor.

In some embodiments, the invention encompasses exposing the cells to an arginase inhibitor, thereby blocking the arginase directly. For example, the inhibitor may permanently inactivate arginase, for example by reacting covalently with a relevant part of the protein, such as the active site. Arginase inhibitors have been previously disclosed, for example in the documents cited in the introduction of the present application.

Preferably, said cells are modified by genetic means and/or at the level of the gene or its expression, so as to prevent arginase expression genetically.

In a preferred embodiment, said arginase activity is impaired by impairing expression of said arginase, for example by
mutating, truncating or deleting a gene encoding said arginase,
administering, mutating, truncating or deleting a gene encoding a transcription factor for said gene encoding said arginase,
administering a nucleotide sequence encoding or comprising a nucleotide sequence capable of binding to an mRNA encoding said arginase. For example, this step may comprise administrating a nucleic acid molecule comprising a nucleotide sequence encoding an mRNA encoding said arginase or binding to said mRNA.

For the purpose of the present specification, a truncation and a deletion of a gene are encompassed by the term “mutation” and its various grammatical forms, such as “mutating”. The term “mutation” further encompasses point mutations, point deletions (deletion of a single nucleotide in the coding region of the gene) and the insertion or deletion of a stretch of nucleotides in a coding region of the gene, for example. More generally, the term “mutation” thus encompasses any genetic alteration resulting in a gene expression that differs from an expression in a cell that does not carry the mutation. For the sole reason of clarity, it is mentioned that some mutations may not result in an altered gene expression, and such silent mutations are generally not considered mutations suitable to impair arginase expression.

For example, the cells may be modified in that the gene or genes encoding arginase, in particular arginase 2, are deleted. For example, the cells may be treated so as to become double knockout with respect to arginase 1 and/or 2 (Arg1^{-/-} and/or Arg2^{-/-}). Mutations in the gene encoding arginase, or in a gene otherwise involved in the control of arginase expression, such as a transcription factor, may be conducted by site directed mutagenesis, for example. Numerous methods are available for site directed mutagenesis, such as Kunkel's method, cassette mutagenesis or PCR site-directed mutagenesis. In an embodiment, arginase encoding genes or transcription factors may be modified by way of gene editing, for example by using the CRISPR/Cas9 technology.

The arginase expression may be impaired by promoting expression of a repressor protein of the arginase gene, for example by transfecting the immune cells with a vector constructed to produce such a repressor protein when inserted in the cells, or by inserting a gene encoding a suitable repressor by way of gene editing technology.

In a preferred embodiment, said arginase expression is reduced by way of RNA interference (RNAi). In an embodiment, said impaired arginase activity is due to administration of a nucleic acid molecule capable of binding to an mRNA encoding said arginase in said immune cells, or administration of a vector or expression system encoding such a nucleic acid molecule. The administration of said nucleic acid molecule or said vector is preferably ex vivo, that is, in isolated cells, for example cells extracted from an individual.

In an embodiment, the method of the invention comprises administering or transcribing RNA that inhibits gene expression or neutralizing mRNA of arginase. For example, the small interfering RNA (siRNA), micro RNA (miRNA) or short hairpin RNA (shRNA) may be administered to the cells, or a vector encoding any one selected from such interfering RNAs. These molecules may be delivered to the cells by way of a suitable delivery system and/or vectors, capable of delivering the interfering RNA inside the cells, or capable of transcribing such interfering RNAs when transferred into the cells.

In an embodiment, the method of the invention comprises administering or transcribing nucleic acid molecules, such as RNAs, that specifically inhibit gene expression or neutralize mRNA of arginase. For example, specifically tailored small interfering RNAs (siRNAs), micro RNAs (miRNAs) or short hairpin RNA (shRNAs) may be administered to the cells, or a vector encoding any one selected from such inhibitory RNAs. These molecules may be delivered to the cells by way of a suitable delivery system and/or vectors, capable of delivering the inhibitory RNAs inside the cells, or capable of transcribing such inhibitory RNAs when transferred into the cells.

In an embodiment, specificity is provided by complementary base pairing of the interfering and/or inhibiting nucleic acid molecule only to an mRNA encoding arginase but not to other mRNA molecules encoding other proteins, and/or only to an RNA molecule regulating arginase expression but not to RNA molecules regulating the expression of other proteins, in particular in said immune cells.

In an embodiment, said inhibitory and/or interfering nucleic acid molecule, preferably RNA, is not microRNA-155 and/or does not comprise microRNA-155.

In other embodiments, for example where arginase expression and/or neutralization of mRNA of arginase is non-specifically inhibited and/or interfered with, said inhibitory and/or interfering nucleic acid molecule may comprise and/or essentially consist of microRNA-155.

In a preferred embodiment, a vector is used that expresses and/or transcribes inhibitory and/or interfering RNAs directly in the cells. Such vectors allow long term gene specific silencing. Such vectors, for example for silencing arginase 1 or arginase 2, may be obtained commercially, for example from Santa Cruz Biotechnology, Inc., USA, which offers a complete line of RNAi Gene Silencers, including siRNA, shRNA Plasmid and shRNA Lentiviral products. Accordingly, a plasmid or vector encoding the interfering RNA may be administered, or the interfering RNA may be directly introduced into the cells, for example using a suitable delivery vehicle. In accordance with another example, a virus particle may be used to administer a shRNA encoding plasmid. Sequences of exemplary siRNA molecules specific to human arginase 2 mRNA, can be derived from the RNA or DNA sequence of arginase 2, disclosed at GenBank at Accession No. NM_001172, for example. SiRNA molecules are also disclosed in Setty B A, et al. Hypoxic Proliferation of Osteosarcoma Cells Depends on Arginase II, Cell. Physiol. Biochem. 39 (2), 802-813 (2016).

In accordance with the invention, the reduction of arginase activity and/or expression may be constitutive or may be inducible. In some embodiments, the reduction of arginase activity is inducible, that is, may be triggered by an external factor that can be controlled, for example by the staff conducting the modification of the cells for adoptive cell transfer. The reduced arginase expression may be rendered inducible by the presence of a suitable promoter, for example on the vector encoding the interfering RNA. Thanks to the promoter, gene silencing occurs only once the promoter is activated. The promoter may be selected, for example, to be activatable by a particular small molecule, which may be separately administered, to the cells or to the patient having received the cells, for example.

On the other hand, by using a promoter that is constitutively active, silencing RNA may be expressed constitutively, resulting in constitutive impairment of arginase expression.

Furthermore, reduction of arginase activity may be transient or stable. Transient or stable impairment of arginase expression may be determined by the choice of the interfering RNA and the way of treatment of the cells. For example, by transfecting cells directly with the appropriate siRNA, arginase expression may be silenced transiently. By transfection with a vector encoding the interfering RNA under the control of a promoter, arginase expression may be silenced stably (in an inducible or constitutive manner) in the cells.

In some embodiments, the immune cells comprise further advantageous features and/or functionalities for treating cancer. Preferably, the cells are further modified to have improved cancer treatment functions. The further modification of the immune cells may be conducted simultaneously as the impairment of the arginase activity as detailed above, or may be conducted in separate, previous or subsequent steps using, for example, the same immune cells. The invention also encompasses that other immune cells are engineered independently so as to have improved cancer treatment functions, wherein these other cells are administered, simultaneously or sequentially with the immune cells of the invention, in a treatment in accordance with the present invention.

In an embodiment, the immune cells are tumor infiltrating lymphocytes (TILs) and/or or the method comprises administrating TILs. Preferably, said TILs are tumor infiltrating T cells. Preferably, said TILs exhibit constitutively or inducibly impaired arginase activity. Preferably, said TILs are administrated to a subject in need thereof, in particular a subject suffering from cancer. Preferably, said TILs have been previously isolated and/or purified from said subject. Preferably, the method comprises impairing arginase activity in said TILs, preferably ex vivo, for example after isolating and/or purifying said TILs and/or before administrating said TILs.

In an embodiment, the immune cells contain and/or express a chimeric antigen receptor (CAR) and/or a transgenic T cell receptor (TCR). In an embodiment, said CAR comprises an antigen binding domain fused via a linker to a T cell signalling domain, in particular a CD3 (zeta) signalling domain. The antigen binding domain preferably comprises one or more variable domains of an antibody. Preferably, said linker comprises a transmembrane domain. When expressed on the surface of CAR cells, the antigen binding domain is on the extracellular side of the cells whereas the signalling domain is on the intracellular side of the cell.

Depending on the design and function of the CAR, the appropriate intracellular domain may be selected. If the extracellular domain is selected so as to recognize (bind to) a target on the cancer cells, the intracellular domain is preferably selected so as to activate the immune cells upon binding of the extracellular domain. Typically, in CAR T cells, binding of the CAR generally results in proliferation of the T cell and production of cytokines, for example. The binding of the CAR T cell to the target site, such as CD19, may directly result in the cancer cell being killed, for example by induction of apoptosis in the cancer cell. The specific effect obtained from binding of the extracellular domain is thus controlled by selection of the appropriate intracellular domain. Typically, CAR immune cells comprise a signalling domain comprising CD3 ζ, and optionally additional signalling domains. The present invention is, however, not limited with respect to the intra- or extracellular domain of a CAR that may be expressed by the immune cell. The person skilled in the art may select the appropriate domains in dependence of the particular cancer cells to be targeted and in dependence of the immune response that is wished to be triggered by the binding.

For example, the antigen binding domain may be specific to B cell antigen CD19. Current adoptive cell transfer therapies (axicabtagene ciloleucel, tisagenlecleucel) against blood cancers, such as leukaemia and lymphomas are based on CAR T cells, in which the antibody variable domain of the CAR is specific to CD19.

Depending on the cancer to be treated, the antibody variable domain of the CAR is preferably specific to a target expressed on the surface of cancer cells. In the art, the following antigen binding domain specificities have been tested for the treatment of the corresponding cancers by CAR therapy. An antigen binding domain specific: to carbonic anhydrase, in particular for a CAR cell therapy against renal cancer; to epidermal growth factor receptor, in particular the variant EGFRvIII CAR for treating glioblastoma; to prostate-specific antigen (PSMA) and/or prostate stem cell antigen (PSCA) in CAR therapy against prostate cancer; to ovarian tumor antigen mucin 16 (MUC16) in the treatment of any one selected from ovarian, fallopian tube and primary peritoneal cancer.

In accordance with embodiments of the present invention, the immune cells are preferably engineered to express one or more CARs, such as a CAR having a specificity selected from the group of antigens mentioned above.

In an embodiment, the immune cells express a CAR and/or or the method comprises administrating immune cells expressing a CAR. Preferably, said CAR expressing cells exhibit constitutively or inducibly impaired arginase activity. Preferably, CAR expressing cells are administrated to a subject in need thereof, in particular to a subject suffering from cancer. Preferably, said immune cells previously isolated and/or purified from said subject and the method comprises the step of treating the cells so as to express said CAR and/or to provide cells previously isolated from a patient or other donor and said cells being previously treated to express said CAR. In an embodiment, the method comprises impairing arginase activity in said CAR expressing cells, preferably ex vivo.

Another modification of the immune cells that is encompassed in accordance with the invention is the switching of internal signalling domains.

In an embodiment, the immune cells comprise and/or express an IL-4 receptor, which is modified or inactivated such as to reduce, annul and/or reverse inhibitory IL-4 signalling triggered by the tumor microenvironment. For example, the signalling domain of the IL-4 receptor may be switched for that of the IL-7 receptor, so as to reverse inhibitory IL-4 signalling.

In an embodiment, the immune cells express two CARs. A first CAR preferably targets the immune cells to particular cancer cells, with an antigen binding domain directed to an antigen, such as PSCA, expressed by the cancer cells. A second CAR may bind, for example, to a synthetic or foreign compound, for example a small-molecule. The compound may be administered to the patient separately and/or together with the CAR cells. The small molecule, when administered binds to the second CAR. When an immune cell is in contact with the target cell, co-stimulation with the compound results in efficient and/or strong activation of the immune cell. T cells co-expressing two CARs, the second binding to rimiducid (a lipid-permeable tacrolimus analogue with homodimerizing activity) are currently tested in phase I trials.

In an embodiment, the immune cells are engineered so as to express an inactive checkpoint protein or so as to lack an inhibitory checkpoint protein. Exemplary immune checkpoint regulators are PD-L1/PD1, CTLA4, B7-H3 (CD276), B7-H4 (B7x/B7S1/VTCN1), HHLA2 (B7H7/B7-H5), VISTA (PD1H, DD1alpha, c10orf54, Gi24, Dies1, SISP1), VSIG, LAG-3, TIGIT, CD96, CD39, CD73, adenosine A2 receptors, CD47, butyrophilins (BTN) and/or TIM-3 (T cell—immunoglobulin—mucin domain).

In an embodiment of the immune cells, the immune response inhibiting signalling domain of a checkpoint protein is inactivated or swapped (replaced) with a co-stimulatory domain. For example, the repressive CD28 domain of PD1 (programmed cell death protein 1) may be mutated so as to be inactive or may be switched with a co-stimulatory domain, for example a CD3 ζ signalling domain or with a CD137 domain, for example.

By rendering the immune checkpoint protein of the immune cells inactive or by transforming the intracellular domain into a stimulatory domain (for example by replacement of the intracellular domain), it is possible to avoid the immune suppressive activity of the tumor cells and/or the tumor microenvironment.

In an embodiment, the immune cells recombinantly express a protein that is capable of inducing apoptosis when triggered by an external factor. This may be seen as a safety measure, allowing the immune cells of the invention to be destroyed in a targeted manner, in case of an undesired side effect noticed following administration of the immune cells to the patient, for example. For example, a signalling domain may be provided in such a manner that binding to the extracellular domain results in apoptosis. The extracellular domain may be binding to an artificial compound, such as a small molecule, as described elsewhere in this specification.

The immune cells may be engineered to express the CAR and/or the transgenic T cell receptor by transfecting the cells with an appropriate vector, or by inserting the genes and/or promoters for expressing the receptor in the genome, for example using gene editing such as CRISPR/Cas9 technology.

For treating cancer, the immune cells are preferably administered to an individual, for example to an individual in need of a cancer treatment. The individual is preferably a patient suffering from cancer. The immune cells may be administered in any suitable way, preferably parenterally. In a preferred embodiment, the immune cells are administered intravenously.

In a preferred embodiment, the immune cells are administered in addition to another cancer treatment. Preferably, the immune cells are used in combination with another cancer treatment. The other cancer treatment may be administered simultaneously and/or separately. Furthermore, the other cancer treatment may be administered separately, in the form of separate compositions, or may be combined in a single composition.

In a preferred embodiment, the immune cells are used and/or administered in combination with a cancer treatment targeting and/or specifically binding to a negative immune checkpoint regulator. Immune checkpoint regulators have been disclosed elsewhere in this specification. In one embodiment, the immune cells may express a cell-surface protein, for example a receptor, binding to the immune checkpoint regulator that may be expressed by cancer cells. For example, as set out above, the immune cells may express a CAR binding to an immune checkpoint regulator protein. In accordance with this embodiment, the entity blocking the immune checkpoint regulator may be expressed in the form of a cell surface protein on the immune cells instead of an unbound/free antibody.

In another embodiment, said (other or additional) cancer treatment comprises an anti-cancer agent and/or molecule, for example an immune checkpoint regulator inhibitor. Preferably, said cancer treatment comprises an antibody, preferably an antibody specifically binding to an immune checkpoint regulator. In a preferred embodiment, said cancer treatment comprises an antibody specifically binding to one or more selected from the group of: PD1, PD-L1, CTLA4, B7-H3, B7-H4, HHLA2, VISTA, VSIG, LAG-3, TIGIT, CD96, CD39, CD73, adenosine A2 receptors, CD47, butyrophilins (BTN) and/or TIM-3.

Antibodies specifically binding immune checkpoint regulators, such as Nivolumab and Pembrolizumab, both of which specifically bind PD-1, are commercially available and are used for treating a variety of different cancers.

Such antibodies may be generally referred to as immune checkpoint regulator inhibitors. The present inventors have observed that the arginase-impaired immune cells of the invention, when combined with a treatment comprising an immune checkpoint regulator inhibitor, results in a synergistic anti-cancer activity. In an embodiment, the antibody specifically binds to an immune checkpoint regulator protein expressed by cancer cells and provided on the surface of the cancer cells. In another embodiment, the antibody specifically binds to a protein, for example a receptor expressed on the surface of the immune cells of the invention, wherein the antibody prevents the protein of the immune cells to get in contact with the corresponding protein expressed on the cancer cell. In a preferred embodiment, the immune checkpoint regulator inhibitor is a monoclonal antibody.

In a preferred embodiment, the immune cells are used in methods of treating and/or preventing cancer. In an embodiment, the cancers selected for treatment are selected from groups comprising leukaemias, lymphomas and/or a solid tumors. One surprising finding of the present inventors is that the immune cells are not only efficient in reducing blood cancer and/or haematological cancers, but are in particular also efficient in reducing the size and/or volume of solid tumors. This is surprising, since the currently used adoptive cell transfer therapies based on CAR T cells are used for treating blood cancers, generally due to the difficulty of obtaining sufficient efficacy towards solid tumors. In current CAR T cell therapies, treatments of solid tumors is accompanied by sometimes important side effects and toxicity problems. The present inventors disclose treatment of solid tumors by adoptive cell transfer in the absence of toxicity side effects.

The present invention is directed to several methods, including methods for treating and/or preventing cancer, immunotherapy methods, methods of cell therapy, methods of improving existing immunotherapy, methods for producing immune cells useful in cancer therapy and prophylaxis, methods for preparing and/or manufacturing a cancer treatment, and/or methods of improving the anti-cancer activity and/or efficacy of immune cells. As detailed in the present specification, the immune cells are preferably treated to have improved anti-cancer activity. More specifically, the cells preferably have increased activity and/or survival in the immune-suppressive environment generated by cancer cells, including cells of solid tumors. Preferably, the immune cells retain their anti-cancer activity and/or function by remaining to some extent inert to at least some of the immune-suppressive activity of cancer cells.

Preferably, the immune cells are treated and/or engineered in order to exhibit improved anti-cancer activity. The treatment preferably results in reduced arginase activity as detailed in this specification. The treatment of the cells preferably takes place ex vivo and/or in vitro, that is, preferably outside the human or animal body. In an embodiment, the method of the invention comprises extracting and/or collecting the yet untreated or wild type immune cells from an individual. In an embodiment, the method of the invention comprises, prior to administering said immune cells, impairing ex vivo arginase activity and/or expression in said immune cells. In an embodiment, the method comprises the administration of the immune cells to a patient.

As disclosed elsewhere in this specification, the immune cells may stem from the patient to be treated, from a donor, or may be derived from culture, for example obtained from stem cells.

The immune cells are preferably provided in the form of a pharmaceutical composition. Preferably, the composition comprises a suitable excipient and/or carrier, for example a solution selected from approximately physiological and/or isotonic solutions, for example saline, in which the cells are suspended. In some embodiments, the pharmaceutical composition may comprise serum, for example obtained from the patient to be treated or from a donor.

The invention also provides a kit for use in methods of treating cancer. The kit preferably comprises an agent suitable for impairing arginase activity and/or expression in immune cells. In some embodiments, the kit comprises an agent capable of inducing RNA interference when administered to the immune cells, wherein said RNAi results in impaired arginase expression. For example, the kit comprises one or more agents as disclosed elsewhere in this specification (siRNA vectors, and so forth). In some embodiments, the kit comprises immune cells, for example off-the-shelf immune cells having impaired arginase activity, e.g. Arg2^{-/-} immune cells.

While certain of the preferred embodiments of the present invention have been described and specifically exemplified above, it is not intended that the invention be limited to such embodiments. Various modifications may be made thereto without departing from the scope and spirit of the present invention, as set forth in the following claims. Herein below, examples of the invention are disclosed. These examples are for illustration only and are not intended to limit the scope of the present invention.

### EXAMPLES

### Example 1: Impaired Tumor Growth and Enhanced Anti-Tumor Immunity in Arg2^{-/-} Mice

For the present examples, Arg2-deficient (double knock-out) mice were used obtained from Charles River Laboratories, Inc. As a first approach, tumor growth in WT and Arg2^{-/-} mice was compared. Two transplantable tumor models were used, the B16 melanoma model and the MC38 colon carcinoma model. In both models, tumor variants expressing ovalbumin (OVA) as a surrogate tumor antigen were used.

In a first experiment, 0.5×10⁶ B16-OVA or MC38-OVA cells were injected s.c. into the back of WT or Arg2^{-/-} mice, and tumor growth was monitored for 2 weeks. N=9, data is pooled from two independent experiments.

In a second experiment, tumor cells were implanted as in the first experiment. 11 (B16-OVA) or 13 (MC38-OVA) days after tumor injection, fluorescently-labeled with high doses of cell tracer violet (CTV^{hi}) OVA-pulsed and low doses)(CTV^{lo}) non-pulsed WT splenocytes were injected i.v. Twenty-four hours later, tumors were excised and cell suspensions from TdLN and ndLN were analyzed by flow cytometry. Specific in vivo killing was calculated as follows: [1−(% CTV^{hi} TdLN/% CTV^{lo} TdLN)/(% CTV^{hi} ndLN/% CTV^{lo} TdLN)]×100.

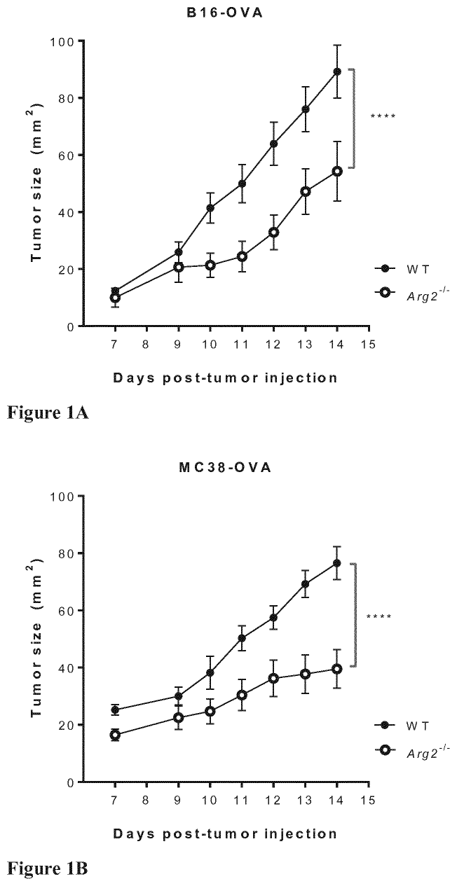

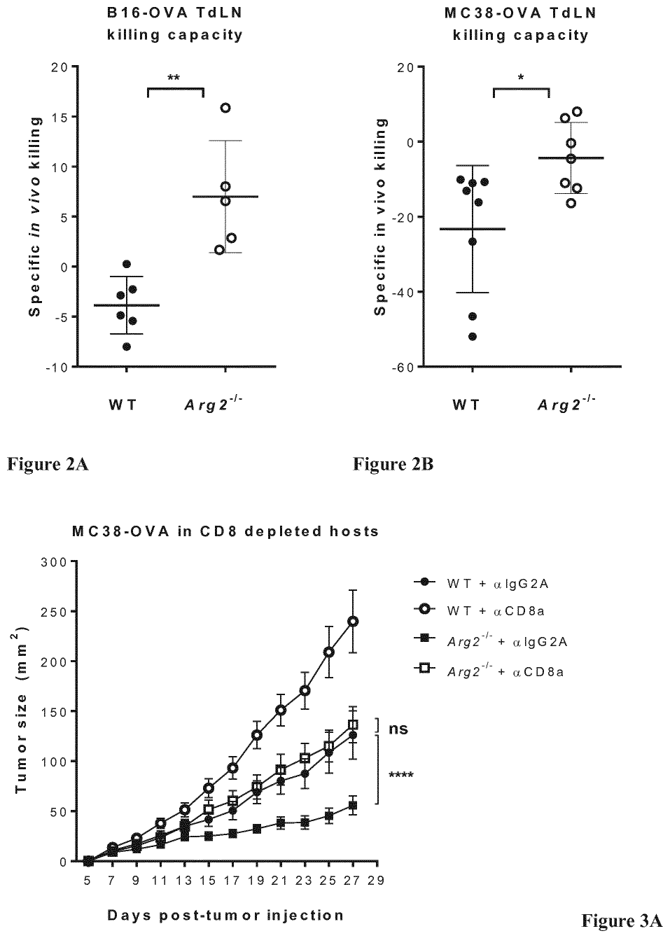

The results are shown in FIGS. 1A, 1B, 2A and 2B (*, p<0.05; **, p<0.01; ****, p<0.0001). As shown in FIGS. 1 A and 1 B, growth of both B16-OVA and MC38-OVA tumors was significantly impaired in Arg2^{-/-} mice. Impaired tumor growth was associated with increased in vivo OVA-specific tumor cell killing in tumor draining lymph nodes (FIGS. 2 A and 2 B). For B16-OVA, a representative of two experiments is shown. For MC38-OVA, data was pooled from two independent experiments.

For further investigations, we focused mainly on the MC38-OVA model.

### Example 2: Contribution of CD8+ T Cells to Control Tumor Growth and Animal Survival

To determine whether impaired MC38-OVA growth in Arg2^{-/-} mice might be due to enhanced control by CD8⁺ T cells, we performed CD8⁺ T cell depletion experiments.

0.5×10⁶ MC38-OVA cells were injected s.c. into the back of WT or Arg2^{-/-} mice, and tumor growth and animal survival was monitored for 4 weeks. CD8⁺ T-cell depletion was performed by several i.p. injections of anti-CD8a⁺ depleting Ab (αCD8a) or IgG2a isotype control Ab (αIgG2A).

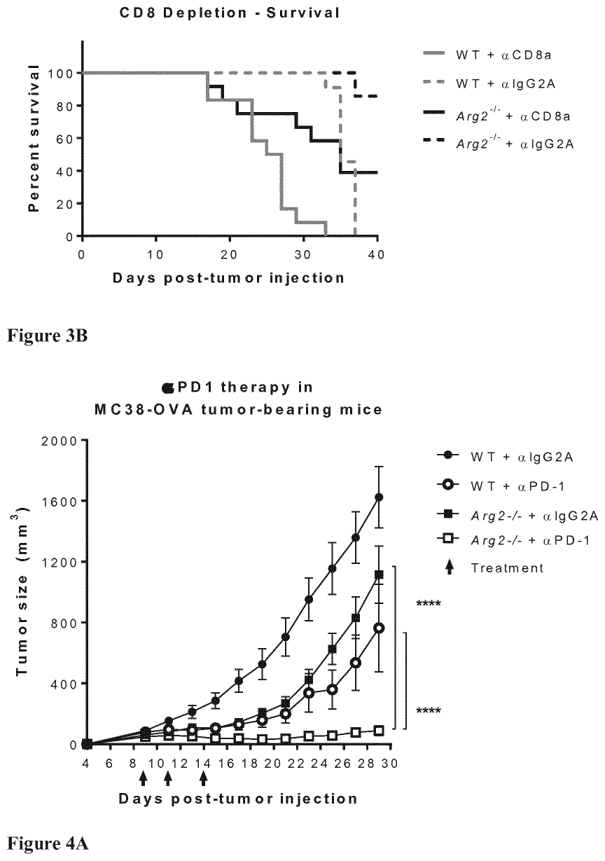

As shown in FIGS. 3A and 3B, reduced tumor growth and increased animal survival, respectively, in Arg2^{-/-} mice was significantly reverted by CD8⁺ T cell depletion, indicating an important role of CD8⁺ T cell-mediated immune control. However, the anti-tumor CD8⁺ T cell response is not the only mechanism at play, as tumor growth in CD8⁺ T cell-depleted Arg2^{-/-} mice was not fully restored to that observed in CD8⁺ T cell-depleted WT mice.

### Example 3: Synergistic Effect of Anti-PD1 Therapy and Arg2-Deficiency on Tumor Growth Inhibition and Animal Survival

MC38 tumors are sensitive to immunotherapy with antibodies that block the T-cell inhibitory PDLL-PD1 checkpoint axis. To determine whether enhanced control of MC38 tumors induced by PDL1-PD1 blockade might collaborate with the mechanism(s) responsible for enhanced control of MC38 tumors resulting from Arg2-deficiency, WT and Arg2^{-/-} mice bearing MC38-OVA tumors were treated with anti-PD1 antibodies.

0.5×10⁶ MC38-OVA cells were injected s.c. into the back of WT or Arg2^{-/-} mice. Mice were injected with anti-PD1 (αPD-1) Ab or IgG2a isotype control Ab (αIgG2A) on days 9, 11 and 14 after tumor injection (green arrows).

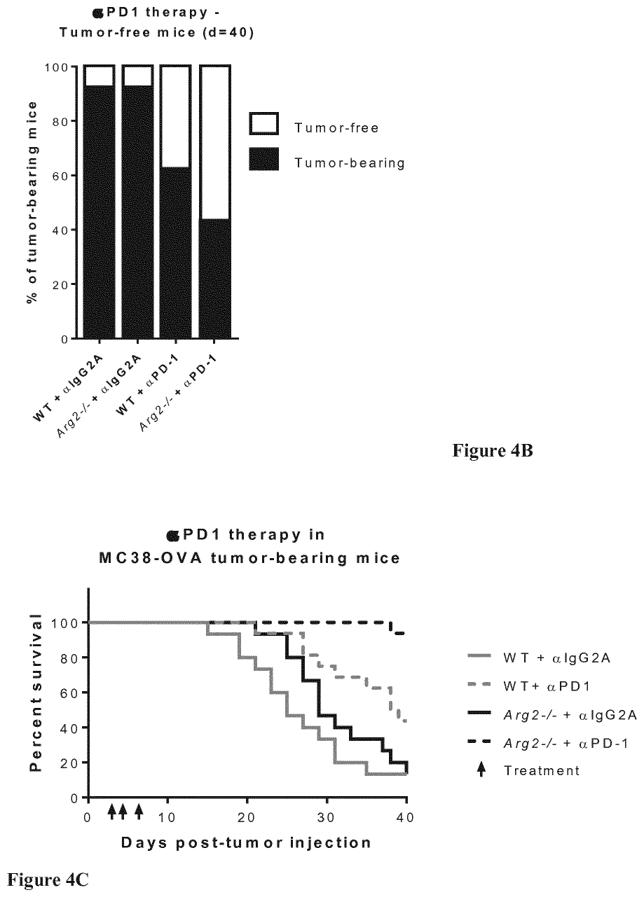

As can be seen from FIG. 4A (data pooled from 2 experiments. ****, p<0.0001), tumor growth in WT mice treated with the anti-PD1 antibody was reduced to an extent similar to reduced tumor growth in untreated Arg2^{-/-} mice. Importantly, treatment of Arg2^{-/-} mice with the anti-PD1 antibody led to an almost complete abrogation of tumor growth. Tumors were actually cleared in many mice, as shown in FIG. 4B. FIG. 4C shows that animal survival was greatly increased in Arg2^{-/-} mice treated with the anti-PD1 antibody. Thus, anti-PD1 therapy and Arg2-deficiency exhibit a strong synergistic effect on tumor growth.

### Example 4: Arg2-Deficiency in BM-Derived Cells is Responsible for Improved Control of Tumor Growth

Reciprocal bone marrow (BM) chimeric mice were generated to determine whether impaired MC38-OVA growth is a consequence of Arg2-deficiency in BM-derived cells or cells of non-hematopoietic origin. WT and Arg2^{-/-} mice were sub-lethally irradiated to destroy the host BM. Hematopoiesis was then reconstituted by transplantation with BM cells from WT or Arg2^{-/-} mice in all four pairwise combinations.

0.5×10⁶ MC38-OVA cells were injected s.c. into the back of the BM chimeric mice, and tumor growth was monitored for 4 weeks. N=11 mice.

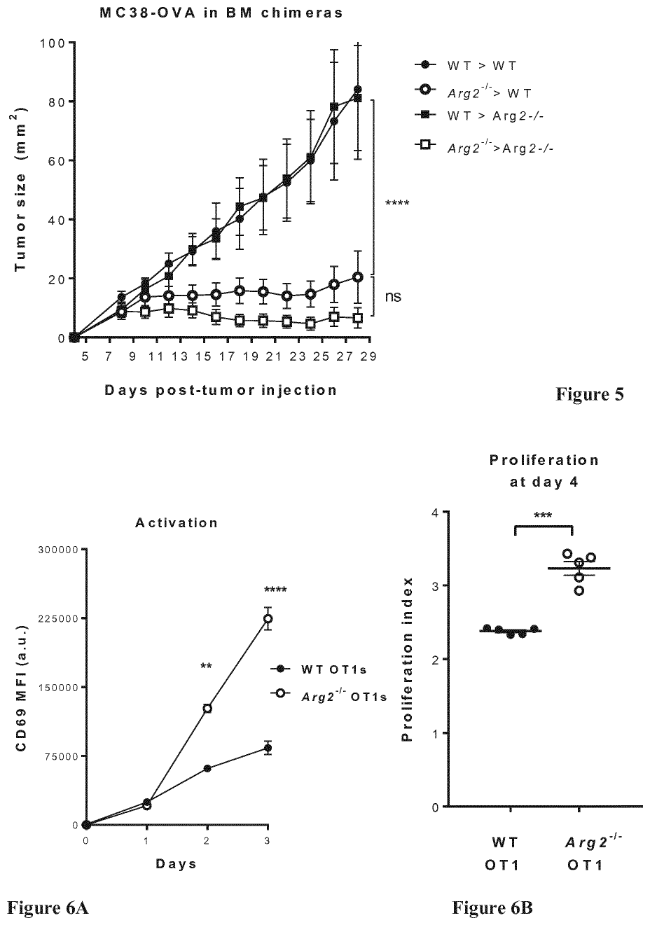

As can be seen from FIG. 5 (data is pooled from 3 independent experiments. ****, p<0.0001), chimeras receiving BM cells from Arg2^{-/-} mice (Arg2^{-/-} >WT and Arg2^{-/-} >Arg2^{-/-}) exhibited strongly reduced tumor growth compared to chimeras receiving BM cells from WT mice (WT>WT and WT>Arg2^{-/-}). These results indicate that reduced tumor growth is due primarily to Arg2-deficiency in BM-derived cells.

### Example 5: Arg2^{-/-} OTI Cells Exhibit Enhanced Activation and Proliferation In Vitro

To determine whether Arg2-deficiency in CD8⁺ T cells might be responsible for improved control of tumor growth, we crossed the Arg2 mutation into OTI mice to obtain double homozygous Arg2^{-/-} OTI mice.

OTI mice have only OVA-specific CD8⁺ T cells because they express a transgene encoding an MHC class I-restricted OVA-specific TCR. This allowed us to compare the functional properties of Arg2^{+/+} and Arg2^{-/-} OVA-specific T cells.

WT OTI or Arg2^{-/-} OTI T-cells were isolated from the crossed mice and were activated in vitro with anti-CD3 and anti-CD28 Abs, cultured in RPMI in 96-well plates, and assessed for activation (CD69 staining) at days 1, 2 and 3 following activation. T cell proliferation (Carboxyfluorescein succinimidyl ester (CFSE) dilution) was determined at day 4 following activation.

The results are shown in FIGS. 6A and 6B (data is representative of 2 independent experiments. **, p<0.01; ***, p<0.001).

In vitro T-cell activation assays shown in FIGS. 6 A and 6 B demonstrated that Arg2^{-/-} OTI cells exhibit increased activation and proliferation compared to Arg2^{+/+} OTI cells.

### Example 6: OTI T Cell Therapy of MC38-OVA Tumors

An in vivo system was developed to compare the control of MC38-OVA tumor growth and animal survival by Arg2^{+/+} and Arg2^{-/-} OTI cells in a WT background.

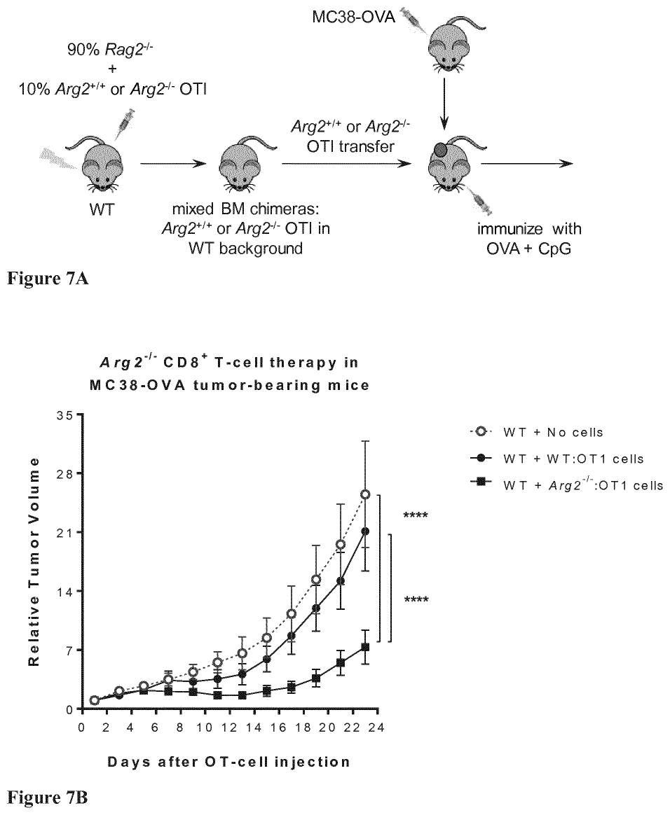

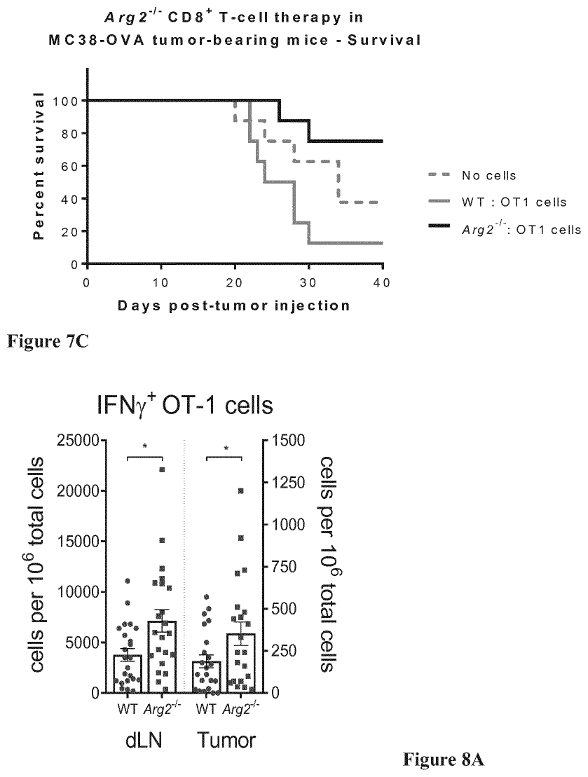

The generation of the in vivo system is illustrated in FIG. 7A. First, mixed BM chimeras were generated, in which irradiated WT mice were reconstituted with a 9:1 mixture of BM derived from Rag2^{-/-} mice and BM cells derived from Arg2^{+/+} or Arg2^{-/-} OTI mice. In these mixed BM chimeras, Arg2^{-/-} and Arg2^{+/+} OTI T cells develop in an environment exhibiting normal Arg2 expression, such that any difference in their functional properties can be attributed to a cell-intrinsic difference in Arg2 expression. It should be noted that B cells derived from the Arg2^{-/-} and Arg2^{+/+} OTI BM will of course also differ with respect to their Arg2 status, but this is unlikely to have any impact because inspection of the ImmGen consortium data indicates that B cells do not express Arg2 mRNA.

The mixed BM chimeras were used as donors of Arg2^{+/+} and Arg2^{-/-} OTI T cells, which were adoptively transferred into WT mice bearing MC38-OVA tumors. More specifically, 10⁶ splenic and lymph node Arg2^{+/+} or Arg2^{-/-} OTI T cells were transferred from the mixed BM chimeras into WT recipients that had been injected 5 days previously with 0.5×10⁶ MC38-OVA tumor cells.

Tumor bearing recipients were then immunized with CpG-B+OVA1 peptide one day after OTI T cell transfer, and tumor growth was monitored.

The results are shown in FIGS. 7B & 7C. Tumor growth was markedly reduced (FIG. 7B) and animal survival was increased (FIG. 7C) in mice that had received Arg2^{-/-} OTI T cells compared to mice that had received no OTI cells or Arg2^{+/+} OTI cells. This experimental setup provided a formal demonstration that Arg2^{-/-} OTI T cells are better equipped than Arg2^{+/+} OTI cells for controlling the growth of MC38-OVA tumors in a WT environment.

### Example 7: Arg2^{-/-} OTI Cells Exhibit Enhanced IFNγ Production, are Less Exhausted and Persist Longer in MC38-OVA Tumor Bearing Animals

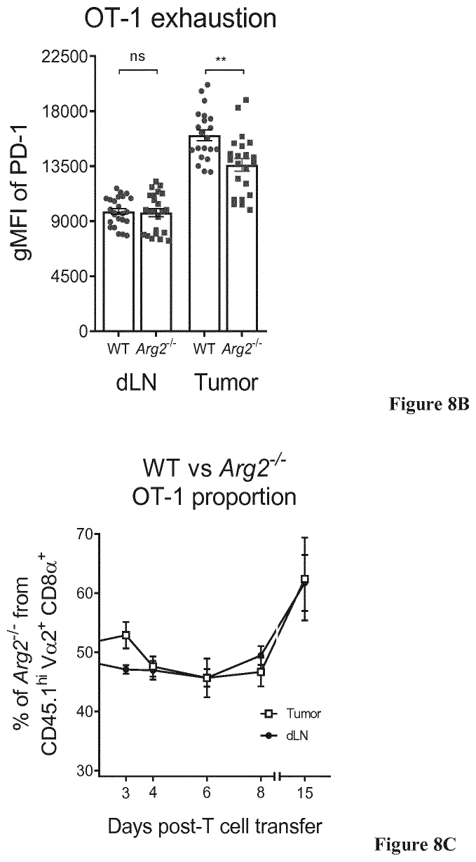

To further investigate the impact of Arg2-deficiency on the effector function of CD8+ T cells in vivo, equal numbers of naïve OTI and Arg2^{-/-} OTI CD8⁺ T cells were transferred into MC38-OVA tumor-bearing WT mice. Tumor bearing recipients were then immunized with CpG-B+OVA-1 peptide one day after OTI transfer, and 7 days post-immunization the OTI cells in draining LNs (dLN) and tumor were analysed by flow cytometry. Frequencies of IFNγ⁺ cells were greater in Arg2^{-/-} OTI cells in both the dLNs and tumors (FIG. 8A). Levels of PD-1 expression were significantly lower on Arg2^{-/-} OTI cells in the tumors (FIG. 8B).

To investigate the impact of Arg2-deficiency on the spatiotemporal dynamics of tumor specific T cell responses, equal numbers of naïve OTI and Arg2^{-/-} OTI CD8⁺ T cells were transferred into MC38-OVA tumor-bearing mice, and their distribution in the hosts was assessed by flow-cytometry in dLN and tumor at different time points after OVA₂₅₇₋₂₆₄ immunization. The CD45.1 marker was used to distinguishing between OTI (CD45.1^{+/+}) and Arg2^{-/-} OTI (CD45.1^{+/-}) cells. By day 15, Arg2^{-/-} OTI cells were markedly more frequent than OTI WT cells in both the dLN and tumors (FIG. 8C), suggesting that they mount a more persistent anti-tumor response.

### Example 8: T-Cell Intrinsic Arg2-Deficiency Synergizes with PD-1 Blockade

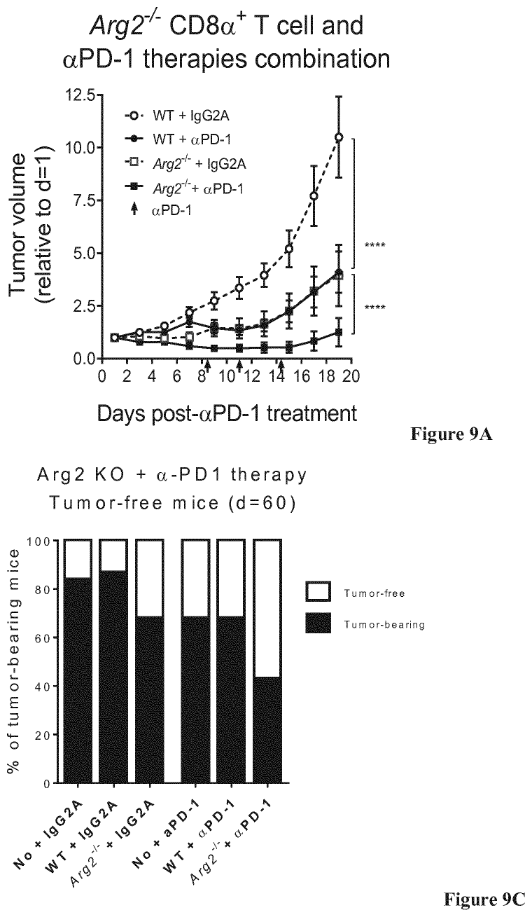

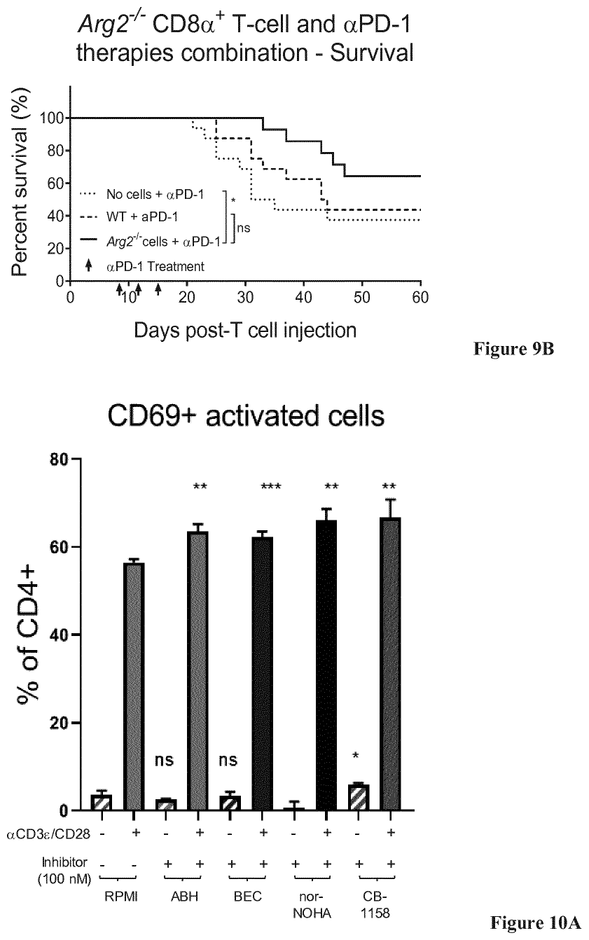

We explored the benefit of combining adoptive Arg2^{-/-} OTI transfer and PD1 blockade in WT MC38-OVA tumor-bearing mice. WT mice were challenged with MC38-OVA tumors and five days later, when tumors were palpable, mice received adoptive transferred cells as illustrated in FIG. 7A except that donors were not chimeric mice, and were immunized next day. At days 8, 11 and 14 after T cell transfer, mice received 200 μg of the relevant antibody, via i.p. injections. In this setting, we again observed synergy between the T cell intrinsic Arg2 deficiency and PD1 blockade. Compared to mice that had received only Arg2^{-/-} OT-I cells or anti-PD1 antibodies, those that were submitted to the combined treatment exhibited a stronger reduction in tumor growth (FIG. 9A), prolonged survival (FIG. 9B) and increased tumor clearance (FIG. 9C).

### Example 9: ARG Inhibition Increases Human T Cell Activation In Vitro

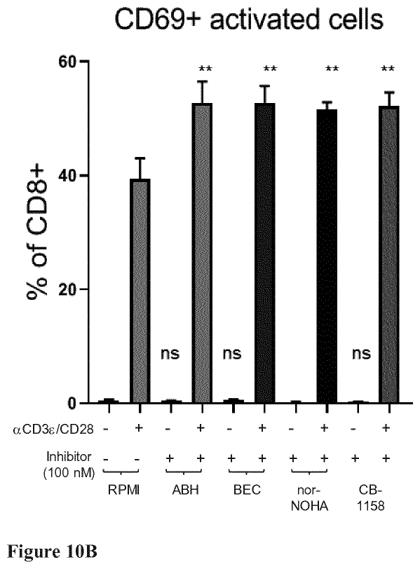

We next determined whether ARG inhibition in human CD4⁺ and CD8⁺ T cells might affect their activation in vitro. Human T cells purified from PBMCs were left untreated or in vitro activated with anti-CD3 and anti-CD28 Abs, cultured in RPMI in 96-well plates in the presence or absence of ARG inhibitors, and assessed for activation (CD69 staining) 24 h post-activation Inhibition of ARG enzymatic function increased the frequency of CD69⁺ cells within CD4⁺ (FIG. 10A) or CD8⁺ T cells (FIG. 10B) demonstrating that ARG inhibition increases human T cell activation in vitro.

1-20. (canceled)

21. A method for treating cancer, the method comprising administration, to a subject in need thereof, of immune cells having impaired arginase activity and/or expression.

22. The method of claim 21, which comprises adoptive cell transfer.

23. The method of claim 21, which comprises: impairing arginase activity and/or expression in said immune cells ex vivo.

24. The method of claim 21, wherein said immune cells have been previously collected from an individual, for example from the subject to be treated.

25. The method of claim 21, wherein said impaired arginase activity and/or expression is impaired arginase 2 activity and/or expression.

26. The method of claim 21, for treating one or more cancers selected from the group consisting of leukaemias, lymphomas and solid tumors.

27. The method of claim 21, in which said arginase activity is impaired by impairing expression of said arginase, for example by:
mutating, truncating or deleting a gene encoding said arginase,
administering, mutating, truncating or deleting a gene encoding a transcription factor for said gene encoding said arginase, and/or
administering a nucleotide sequence encoding or comprising a nucleotide sequence capable of binding to an mRNA encoding said arginase.

28. The method of claim 21, in which said arginase activity is impaired by exposure of said cells to an ex vivo treatment for impairing said arginase activity and/or expression.

29. The method of claim 21, wherein said impaired arginase activity is due to ex vivo administration of a nucleic acid molecule capable of binding to an mRNA encoding said arginase in said immune cells, or administration of a vector encoding such a nucleic acid molecule.

30. The method of claim 21, wherein said immune cells are selected from the group consisting of T cells, natural killer cells (NK cells), innate lymphoid cells and dendritic cells.

31. The method of claim 21, wherein said immune cells are CD3⁺ and/or CD4⁺ and/or CD8⁺ T cells.

32. The method of claim 21, wherein said immune cell further contains a chimeric antigen receptor (CAR) and/or a transgenic T cell receptor, optionally wherein said CAR comprises an antigen binding domain fused via a linker to a T cell signalling domain, optionally in a CD3 ζ signalling domain.

33. The method of claim 21, wherein said immune cells are administered in combination with a cancer treatment targeting and/or specifically binding to a negative immune checkpoint regulator.

34. The method of claim 33, wherein said cancer treatment targets the immune checkpoint regulators PD-L1/PD1, CTLA4, B7-H3 (CD276), B7-H4 (B7x/B7S1/VTCN1), HHLA2 (B7H7/B7-H5), VISTA (PD1H, DD1alpha, c10orf54, Gi24, Dies1, SISP1), VSIG, LAG-3, TIGIT, CD96, CD39, CD73, adenosine A2 receptors, CD47, butyrophilins (BTN) and/or TIM-3 (T cell-immunoglobulin-mucin domain 3).

35. The method of claim 33, wherein said cancer treatment comprises an antibody specifically binding to one or more selected from the group consisting of: PD1, PD-L1, CTLA4, B7-H3, B7-H4, HHLA2, VISTA, VSIG, LAG-3, TIGIT, CD96, CD39, CD73, adenosine A2 receptors, CD47, butyrophilins (BTN) and TIM-3.

36. An isolated and/or purified immune cell having constitutively or inducible impaired arginase activity and/or expression.

37. The isolated and/or purified immune cell of claim 36, in which said arginase activity is impaired by impairing expression of said arginase, for example by:
mutating, truncating or deleting a gene encoding said arginase,
administering, mutating, truncating or deleting a gene encoding a transcription factor for said gene encoding said arginase, and/or
administering a nucleotide sequence encoding or comprising a nucleotide sequence capable of binding to an mRNA encoding said arginase.

38. The isolated and/or purified immune cell of claim 36, which is selected from the group consisting of T cells, natural killer cells (NK cells), innate lymphoid cells and dendritic cells.

39. The isolated and/or purified immune cell of claim 36, which is CD3⁺ and/or CD4⁺ and/or CD8⁺ T cells.

40. The isolated and/or purified immune cell of claim 36, which further contains a chimeric antigen receptor (CAR) and/or a transgenic T cell receptor, optionally wherein said CAR comprises an antigen binding domain fused via a linker to a T cell signalling domain, optionally in a CD3 ζ signalling domain.

41. A method for improving the anti-cancer activity of immune cells for adoptive cell transfer, the method comprising: impairing arginase activity and/or expression in said immune cells ex vivo.
